(19) United States
(12) Patent Application Publication
(10) Pub. No.: US 2023/0329569 A1
(43) Pub. Date: Oct. 19, 2023

(54) System And Method For Multi-Node PPG On Wearable Devices

(71) Applicant: Google LLC, Mountain View, CA (US)

(72) Inventors: Sherk Chung, Berkeley, CA (US); Saket Patkar, Mountain View, CA (US); Ian Atkinson, Castro Valley, CA (US)

(21) Appl. No.: 18/025,585

(22) PCT Filed: Oct. 20, 2020
(86) PCT No.: PCT/US2020/056509

§ 371 Date: Mar. 9, 2023

Related U.S. Application Data
(60) Provisional application No. 63/076,138, filed on Sep. 9, 2020.

(51) Int. Cl. A61B 5/024; A61B 5/00

(52) U.S. Cl. CPC A61B 5/02416; A61B 5/681; A61B 5/6898; A61B 5/7239; A61B 2562/0219

(57) ABSTRACT

Estimates related to heart rate or blood oxygen levels from wearable PPG devices can be exchanged between the devices. Estimated heart rates can be combined at either of the PPG devices or another device using a weighted average to derive a more accurate heart rate or blood oxygen level. A single PPG device is susceptible to motion artifacts and other sources of noise creating inaccurate readings. A time variable weighted average of the two heart rate estimates from two wearable PPG devices provides a real-time heart rate which is more accurate and less susceptible to error. Further, derivative metrics, which analyze the underlying heart rate estimate signals or blood oxygen levels, can also be combined based on rules to create a more accurate derivative metric. Notifications related to the heart rate estimate and derivative metrics can be provided to a user on a user device.

### CROSS-REFERENCE TO RELATED APPLICATIONS

The present application claims the benefit of the filing date of U.S. Provisional Application No. 63/076,138, filed Sep. 9, 2020, entitled System And Method For Multi-Node PPG On Wearable Devices, the disclosure of which is hereby incorporated herein by reference.

### BACKGROUND

Photoplethysmography (PPG) is an optical measurement method which measures changes in blood volume and requires a light source and a photodetector. A photodetector, typically placed at or close to the surface of skin, detects light which is either transmitted through or reflected from vascular tissue to the photodetector. This light corresponds to measuring variations in the volume of blood circulation, which can be used to monitor heart rate. The change in volume caused by a pulse or cardiac cycle can be measured as a peak or trough in the intensity of light. The technique can also be used to measure other aspects related to blood flow, such as oxygen saturation level of the blood.

Heart rate computation using PPG involves emitting light from a light source such as an LED and taking sensor readings from a photodiode that is placed in contact or in very close proximity to a user's skin. The emitted light penetrates inside the skin, and blood pulsing through the tissue affects the amount of light that is reflected and diffused by the user's tissue. The photodiode measures the light intensity in the tissue and an algorithm translates the variation in intensity to a computed heart rate (HR).

The accuracy of the computed heart rate is typically proportional to the signal-to-noise ratio (SNR) of the PPG signal. Accuracy can be improved by using a stronger light source, or by reducing noise in the system. For some devices, such as smart watches for example, power constraints limit use of available techniques to improve SNR. Such techniques can include for example increasing power to the LED or increasing the PPG sensor sampling rate.

Further, despite an increase in SNR of the PPG signal at the expense of increasing power, other degradations or sources of error can be introduced which may impact the accuracy of the measured parameters. For example, “motion artifacts” can be introduced into the PPG signal owing to physical motion of a user affecting the signal rather than a change in the heart rate itself. For example, when a person is wearing a wrist band with a PPG sensor, the normal movement of the arm, such as swinging the arm, can introduce artifacts in the PPG signal which can be an order of magnitude larger than the artifacts related to the heart rate. Such motion artifacts degrade the accuracy of common wearable PPG systems, and can furthermore introduce false positive readings where the algorithm produces erroneous results based on motion artifacts.

### SUMMARY

Aspects of the present disclosure include an apparatus, the apparatus comprising a processing device coupled to a memory storing instructions, the instructions causing the processing device to receive a first heart rate estimate signal from a first wearable device having a first sensor that can generate a first signal associated with a human heart rate, receive a second heart rate estimate signal from a second wearable device having a second sensor that can generate a second signal associated with the human heart rate, and combine the first heart rate estimate signal and the second heart rate estimate signal to generate a third heart rate estimate. The processing device can be configured to receive blood oxygen levels. The first sensor and the second sensor can be photoplethysmography (PPG) sensors. The first wearable device and the second wearable device can contain an accelerometer. The first heart rate estimate signal and the second heart rate estimate signal can be calculated by the first wearable device and the second wearable device based upon data from PPG sensors and accelerometers. The processing device can be configured to wirelessly receive either the first heart rate estimate signal or the second heart rate estimate signal. The processing device can be included in the first wearable device. The second wearable device can wirelessly transmit the second heart rate estimate signal to a first wireless interface included on the first wearable device. The first wearable device and the second wearable device can be either a smartwatch or an earbud. The third heart rate estimate can comprise a weighted combination of the first heart rate estimate signal and the second heart rate estimate signal. The processing device can be a smartphone. A display can be coupled to the processing device. The processing device can be configured to notify the user of a third heart rate estimate using the display. The processing device can be configured to generate a third metric based upon a first metric and a second metric, the first metric and the second metric being generated at the first wearable device and the second wearable device respectively, and the first metric and the second metric being derived using a derivative algorithm. The derivative algorithm can take as inputs either the first heart rate estimate signal or the second heart rate estimate signal and output either the first metric or the second metric. The third metric can be used for one of: atrial fibrillation detection, heart regularity, sleep analysis, emotional measurement, menstrual cycles tracking, respiration tracking, illnesses detection, or hydration levels. The third metric can be based on a weighted combination of the first metric and the second metric, or on more sophisticated fusion techniques such an optimizing control loop or multivariate kalman filter. The weights can be based on a confidence level associated with the first and second heart rate signal.

Additional aspects of the disclosure include a method of determining a physical condition of a user, the method comprising receiving, from a first device, a first measure of a heart rate or a blood oxygenation level of the user; receiving, from a second device, a second measure of the heart rate or the blood oxygenation level of the user; generating, at a processing device, a third measure of the heart rate or the blood oxygenation level of the user based on at least a combination of the first measure and the second measure of the heart rate or the blood oxygenation level of the user; wherein the first measure of the heart rate or the blood oxygenation level is generated at the first device and is based on at least a first signal received by a sensor of the first device and the second measure of the heart rate or the blood oxygenation level is generated at the second device and is based on at least a second signal received by a sensor of the second device. The user can be notified of the third measure. The first measure or the second measure can be received by the first device or second device, respectively, in real time. The third measure can be generated in real time. The processing device can be neither the first device nor the second device. The processing device can be either the first device or the second device. The processing device can apply weights to the first measure or the second measure to generate the third measure. The first device and the second device can be configured to generate a first derivative metric related to the user and a second derivative metric related to the user respectively using a derivative algorithm. The first metric or the second metric can be used to determine one of atrial fibrillation detection, heart regularity, sleep analysis, emotional measurement, menstrual cycles tracking, respiration tracking, illnesses detection, or hydration levels. The derivative algorithm can take as input the first measure or the second measure and provide as output the first derivative metric or the second derivative metric. The processing device can be configured to generate a third derivative metric related to the user based upon at least the first derivative metric and the second derivative metric. The third metric must include or use the first metric and second metric in its computation. An alert can be provided to the user upon the third metric being generated.

Additional aspects of the disclosure include a system, the system comprising a first wearable device having a first wireless interface and a first sensor, the first wearable device configured to generate a first heart rate estimate signal associated with a human heart rate; a second wearable device having a second wireless interface and a second sensor, the second wearable device configured to generate a second heart rate estimate signal associated with a human heart rate and; and a processing device coupled to a memory storing instructions, the instructions causing the processing device to receive the first heart rate estimate signal from the first wearable device, receive the second heart rate estimate signal from a second wearable device, and combine the first heart rate estimate signal and the second heart rate estimate signal to generate a third heart rate estimate. The first sensor and the second sensor are photoplethysmography (PPG) sensors. The first heart rate estimate signal and the second heart estimate rate signal are calculated by the first wearable device and the second wearable device based upon data from the PPG sensors respectively. The processing device can be included in the first wearable device and receive the second heart estimate rate signal using the first wireless interface included on the first wearable device. The second wearable device can wirelessly transmit the second heart estimate rate signal to the first wireless interface. The processing device can comprise a smartphone. The first wearable device and the second wearable device can comprise either a smartwatch or an earbud.

### BRIEF DESCRIPTION OF THE DRAWINGS

The accompanying drawings are not intended to be drawn to scale. Like reference numbers and designations in the various drawings indicate like elements. For purposes of clarity, not every component may be labeled in every drawing. In the drawings:

FIG. 1A is a schematic view of a PPG module according to aspects of this disclosure.

FIG. 1B is a schematic drawing of electronics according to aspects of this disclosure.

FIG. 2A is an illustration of a wearable user device capable of PPG functions according to aspects of this disclosure.

FIG. 2B is a diagram of user interfaces according to aspects of this disclosure.

FIG. 2C is a diagram of user interfaces according to aspects of this disclosure.

FIG. 2D is a schematic diagram of communication between devices according to aspects of this disclosure.

FIG. 2E is a schematic diagram of communication between devices according to aspects of this disclosure.

FIG. 2F is a schematic diagram of communication between two PPG modules according to aspects of this disclosure.

FIG. 3 is a flowchart of an example method according to aspects of this disclosure.

FIG. 4 is a flowchart of an example method according to aspects of this disclosure.

### DETAILED DESCRIPTION

Generally, and as non-limiting examples, as used in this disclosure, a “PPG sensor” refers to a photodiode or other sensor which is capable of measuring light. In some examples, the light for the PPG sensor will arrive from an LED or other light source. “PPG data” can generally refer to the readings from a PPG photodiode. A “PPG algorithm” can generally refer to an algorithm that translates or uses PPG data to generate an estimated heart rate. As one non-limiting example, a “PPG system” can generally refer to a combination of a PPG sensor, a CPU or other computing device which can include memory, and a PPG algorithm which can read PPG data and generate an estimated heart rate.

### Overview

The disclosed technology in one aspect may comprise methods, systems, and apparatuses which can be used to improve the accuracy of PPG devices by combining two or more PPG devices to reduce the error in PPG heart output. As explained below, in some examples, each PPG device can be a wearable user device which contains a PPG module. Each PPG device has an inherent error due to numerous possible factors including motion artifacts. The combination of heart rate estimates from more than one PPG device improves the accuracy of the estimate compared to the estimate from a single PPG device.

In other aspects, the disclosed technology enhances the accuracy of heart rate computation through photoplethysmography (PPG). Rather than each PPG device providing “raw” PPG data, each PPG device is configured to compute its own heart rate estimate signals, blood oxygen saturation levels, or other metrics related to a user using its own PPG algorithms. Aspects of the disclosed technology provide for combining two or more heart rate estimate signals, or other metrics related to a user, to generate a third heart rate estimate or third metric. In some examples, the first device and the second device can be worn on different parts of a user's body. For example, the first device can be a wrist-worn device with an embedded PPG system and the second device can be an earbud device with an embedded PPG system. The first and second heart rates can be combined by various fusion techniques. For example, the first and second heart rates can be combined using a weighted average, optimization control loop, or a multivariate kalman filter.

In another aspect, the first and/or second heart rate estimates can be communicated wirelessly to a computing device which may combine the first and second heart rate estimates to produce a third, more reliable and accurate, heart rate estimate associated with a user wearing two or more wearable devices. The wearable devices may include an earbud and a smart watch. The computing device may reside in either device or in some instances may reside in a smartphone or server communicatively coupled to one or each wearable device.

In other aspects, the disclosed technology improves the rate of “false positives” related to information which can be derived from a heart rate estimate, such as for example, atrial fibrillation. By estimating the derivative metric at each PPG device or PPG node of a system, and using a combination of the metrics, the number of false positives related to one metric can be reduced.

In some examples, a first PPG node or PPG device can be configured to send an activation signal to a second PPG node or PPG device upon the first PPG node detecting accelerometer data, motion artifacts larger than a certain predetermined threshold, or a confidence metric related to a measurement being lower than a predetermined threshold. Responsive to the activation signal, the second PPG node can perform PPG readings or PPG algorithms, allowing the overall accuracy of PPG related calculations and algorithms within the system to be improved. In other examples, the activation signal can first be sent to another user device, such as a cell phone, which in turn can cause the activation of a second PPG node or PPG device. A PPG node can be any device which is capable of obtaining a PPG signal and performing PPG related calculations. In some examples, a PPG node or PPG device can be any device that contains a PPG module as described below. In some examples, a PPG node can be a smartwatch, earbud, or other device containing a PPG module.

In some examples, user devices can include PPG nodes and memory with algorithms to compute a derivative metric from a PPG based heart rate using a derivative algorithm. For example, the devices can produce a derivative metric such as the presence of atrial fibrillation. (Afib). Although some methods below are illustrated with respect to the presence of Afib, other derivative metrics based on other derivative algorithms can be improved by using the disclosed technology. For example, non-limiting examples of derivative metrics sleep analysis, emotion measurement, menstrual cycles tracking, respiration tracking, illnesses detection, hydration analysis.

In yet other examples, the examples given below with respect to heart rate can also be applied to other measurements detectable by a PPG module or equivalent module. For example, blood oxygenation (SPO2) can be detected using PPG modules, and all the methods described for PPG with respect to a heart rate estimate or metrics derived therefrom can also be applied using SPO2 instead of heart rate.

### Example Systems

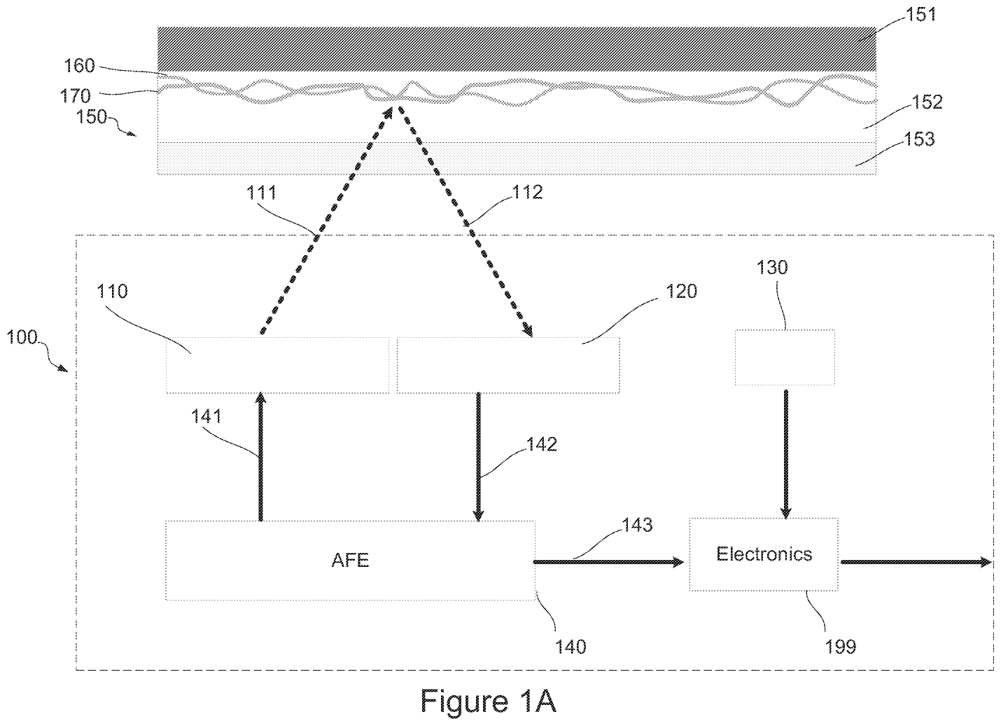

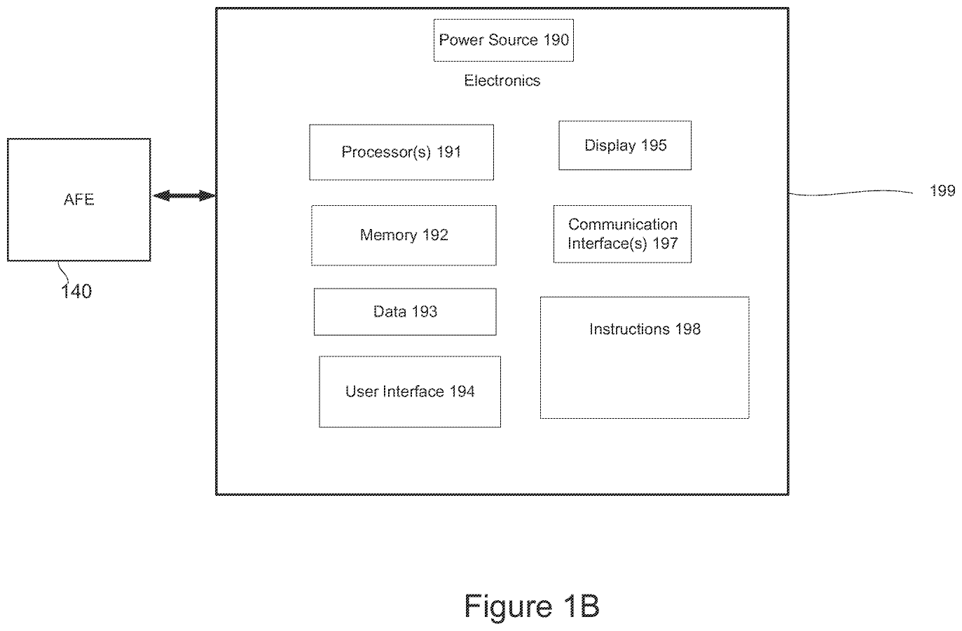

FIG. 1A illustrates a device which can be used to perform PPG, module 100. Module 100 can comprise a light source, such as light source 110, one or more light sensors capable of detecting light, such as photodetector 120, accelerometer 130, analog front end (AFE) 140, and electronics 199. In some examples, electronics 199 may be some or all of the features of electronics 199 described below with reference to FIG. 1B. In other examples, features, operations, or components of AFE 140 and electronics 199 may be exchanged or combined in various permutations.

Rays 111 and 112 are light rays, with the arrow indicating the direction in which the light travels. The light can be incident on a dermis, such as skin 150. Although skin 150 is shown, it is possible that the device is applied to other parts of a human body, such as for example, a nail or soft tissue.

FIG. 1A illustrates a light source 110. One example of a light source is a light-emitting diode (LED). An LED is a semiconductor light source which emits light responsive to electricity flowing through it. Electrons in the semiconductor recombine with electron holes, releasing energy in the form of photons. LEDs can be engineered or chosen to emit light at a particular wavelength or range of wavelengths. In other examples, light source 110 can be made of any commercially available source of light, such as lasers, specially designed semiconductors, incandescent light, electrodeless lamps, or halogen lamps. In other examples, light source 110 can further be made of one or more light sources configured to generate light of different wavelengths, such as an LED configured to generate red light which is close to a wavelength of 660 nm, an LED configured to generated green light which is close to a wavelength of 530 nm. These different light sources may be chosen to measure different aspects of a cardiovascular system when performing PPG. For example, green light may provide information regarding a heartbeat while red light may provide information about blood oxygen saturation, due to the relative absorption and reflection of these colors within the cardiovascular system.

A photodetector, such as photodetector 120, can be a semiconductor device that converts light into an electrical current. The photodetector can generate a current which is proportional to the number of photons hitting the surface. As electricity is generated when photons are absorbed in the photodetector, the photodetector can act as a sensor for light. The photodetector can be any device which is capable of sensing intensities and/or wavelengths of light. Photodetector 120 can be a photodiode or a photosensor. In some examples, photodetector 120 can be chosen to be more sensitive to specific wavelengths of light. In some examples, photodetector 120 can be chosen or configured to be more sensitive or only sensitive to green light while another photodetector can be configured to be more sensitive or only sensitive to red light. Photodetector 120 can also be made of an array of photodetectors. Additional circuitry, calibration, or electronics can be incorporated into the photodetectors, AFE 140, or electronics 199 to ensure a better signal to noise ratio and reduce the effect of ambient light.

In some examples, readings from photodetector 120 can be converted to digital samples at AFE 140 which are forwarded to a CPU of electronics 199, where a PPG algorithm uses PPG data 143 to generate a heart rate estimate. Peak detection techniques, which can use either a time domain or a frequency domain algorithm, can be used to estimate heart rate from PPG data, but the presence of motion artifacts (MA) can make accurate peak detection challenging. Motion artifacts can occur when a user is not relatively still, causing motion in a portion of the body to change the reflected light being received by photodetector 120. For example, a MA generated when a user is swinging his or her arm can trick PPG algorithms to lock onto an incorrect peak or mask the true peak associated with the heart rate of the user.

AFE 140 can contain an LED driver and an analog-to-digital converter (ADC). An ADC converts an analog signal into a digital signal. An LED driver can “drive” or control light source 110. AFE 140 can be used to drive light source 110 through a drive signal 141. AFE 140 can also receive an analog signal 142 from photodetector 120. In some examples, AFE 140 can be part of electronics 199, or components of electronics 199, described in more detail below, can be included in AFE 140. AFE 140 can generate from the analog signal received PPG data, and transfer this information to electronics 199, through signal 143. Signal 143 can be digital or analog. In some examples, signal 143 is forwarded to a processor within electronics 199.

Accelerometer 130 can be any electromechanical device which is configured to measure acceleration responsive to acceleration forces. Accelerometer 130 can generate vectors reflecting acceleration in one or more independent dimensions. In order to identify peaks created by MA, PPG modules are typically accompanied by an accelerometer. A person of skill in the art will appreciate how data from an accelerometer can be used in conjunction with data from or derived from photodetector 120 in a PPG algorithm in a time-domain adaptive filter to cancel out noise generated by motion. In some examples, accelerometer data can be used in a Fourier transform to identify MA peaks in the frequency domain Despite these techniques, the cancellation of a motion artifact is difficult and has significant effects on the accuracy. It is also known in the art that cancellation of MA is difficult, which results in lower-than-desired accuracy of the heart rate estimates when the user is in motion, particularly for wrist-worn devices like medical bands and smartwatches where a user is more likely to move his or her arm and the amount of blood being transmitted through the veins and arteries is subject to a greater amount of change based on this motion. In addition, derivative algorithms which use the information generated from the PPG-based heart rate would inherit the errors introduced by the MAs. For example, other algorithms that depend on PPG-based heart rate can include energy expenditure, breathing rate, atrial fibrillation, sleep analysis, and stress analysis.

Also illustrated in FIG. 1A is skin 150, with a hypodermis layer 151, a dermis layer 152, and an epidermis layer 153 which may contain vein 160 and artery 170. Light generated from light source 110, such as ray 111, can be emitted from module 100 to skin 150. Some of the light emitted from the light source penetrates the skin and is reflected back to photodetector 120, such as ray 112. The reflected light is used to compute an estimated heart rate. Light that reflects off or is transmitted back from these layers is useful for the purpose of PPG.

Variations in the light received by the photodetector can be used to determine various aspects of a cardiovascular system, such as the heart rate, pulse, oxygen saturation in the blood, or other health-related information. In some examples, a wave form can be derived from the continuous or near-continuous monitoring of light received by photodetector 120. Light source 110 and photodetector 120 can be connected with electronics 199 or AFE 140 to control the emission of light, and to monitor and analyze the light received from skin 150.

It is to be understood that although module 100 is illustrated with a specific configuration, other arrangements of these components are within the scope of this disclosure. In other examples, module 100 can be included or arranged within user devices, such as a mechanical watch, a smart watch, a smart ring, a cell phone, earbud, headphone, armband, or a laptop computer. In other examples, module 100 can be integrated into jewelry, such as a pendant, necklace, bangle, earring, armband, ring, anklet, or other jewelry.

FIG. 1B illustrates additional aspects of electronics 199. Although the description in FIG. 1B is given with respect to electronics 199, a person of skill in the art should understand that in some examples AFE 140 and electronics 199 can be combined or operate collectively. Illustrated in FIG. 1B is a bidirectional arrow indicating that communication between AFE 140 and electronics 199 can occur.

Electronics 199 may contain a power source 190, processor(s) 191, memory 192, data 193, a user interface 194, a display 195, communication interface(s) 197, and instructions 498. The power source may be any suitable power source to generate electricity, such as a battery, a chemical cell, a capacitor, a solar panel, or an inductive charger. Processor(s) 191 may be any conventional processors, such as commercially available microprocessors or application-specific integrated circuits (ASICs); memory, which may store information that is accessible by the processors including instructions that may be executed by the processors, and data. Memory 192 may be of a type of memory operative to store information accessible by the processors, including a non-transitory computer-readable medium, or other medium that stores data that may be read with the aid of an electronic device, such as a hard-drive, memory card, read-only memory (“ROM”), random access memory (“RAM”), optical disks, as well as other write-capable and read-only memories. The subject matter disclosed herein may include different combinations of the foregoing, whereby different portions of the instructions and data are stored on different types of media. Data 193 of electronics 199 may be retrieved, stored or modified by the processors in accordance with the instructions 198. For instance, although the present disclosure is not limited by a particular data structure, data 193 may be stored in computer registers, in a relational database as a table having a plurality of different fields and records, XML documents, or flat files. Data 193 may also be formatted in a computer-readable format such as, but not limited to, binary values, ASCII or Unicode. Moreover, data 193 may comprise information sufficient to identify the relevant information, such as numbers, descriptive text, proprietary codes, pointers, references to data stored in other memories (including other network locations) or information that is used by a function to calculate the relevant data.

Instructions 198 may control various components and functions of PPG module 100. For example, instructions 198 may be executed to selectively activate light source 110 or process information obtained by photodetector 120. In some examples, algorithms can be included as a subset of or otherwise as part of instructions 198 included in electronics 199. Instructions 198 can include algorithms to interpret or process information received, such as information received through or generated by analyzing a ray received at a photodetector, PPG signal 143, or information stored in memory. For example, physical parameters of the user can be extracted or analyzed through algorithms. Without limitation the algorithms could use any or all information about the waveform, such as shape, frequency, or period of a wave, Fourier analysis of the signal, harmonic analysis, pulse width, pulse area, peak to peak interval, pulse interval, intensity or amount of light received by a photodetector, wavelength shift, or derivatives of the signal generated or received by photodetector 120. Other algorithms can be included to calculate absorption of oxygen in oxyhemoglobin and deoxyhemoglobin, heart arrhythmias, heart rate, premature ventricular contractions, missed beats, systolic and diastolic peaks, and large artery stiffness index. In yet other examples, artificial learning or machine learning algorithms can be used in both deterministic and non-deterministic ways to extract information related to a physical condition of a user such as blood pressure and stress levels, from, for example, heart rate variability. PPG can also be used to measure blood pressure by computing the pulse wave velocity between two points on the skin separated by a certain distance. Pulse wave velocity is proportional to blood pressure and that relationship can be used to calculate the blood pressure. In some examples, the algorithms can be modified or use information input by a user into memory of electronics 199 such as the user's weight, height, age, cholesterol, genetic information, body fat percentage, or other physical parameter. In other examples, machine learning algorithms can be used to detect and monitor for known or undetected health conditions, such as an arrhythmia, based on information generated by the photodetectors and/or processors.

User interface 194 may be a screen which allows a user to interact with PPG module 100, such as a touch screen or buttons. Display 195 can be an LCD, LED, mobile phone display, electronic ink, or other display to display information about PPG module 100. User interface 194 can allow for both input from a user and output to a user. In some examples, the user interface 194 can be part of electronics 199 or PPG module 100, while in other examples, the user interface can be considered part of a user device.

Communication interface(s) 197 can include hardware and software to enable communication of data over standards such as Wi-Fi, Bluetooth, infrared, radio-wave, and/or other analog and digital communication standards. Communication interface(s) 197 allow for electronics 199 to be updated and information generated by PPG module 100 to be shared to other devices. In some examples, communication interface(s) 197 can send historical information stored in memory 192 to another user device for display, storage, or further analysis. In other examples, communication interface(s) 197 can send the signal generated by the photodetector to another user device in real-time or afterwards for display on that device. In other examples, communication interface(s) 197 can communicate to another PPG module. Communication interface(s) 197 can include bluetooth, Wi-Fi, Gazelle, ANT, LTE, WCDMA, or other wireless protocols and hardware which enable communication between two devices.

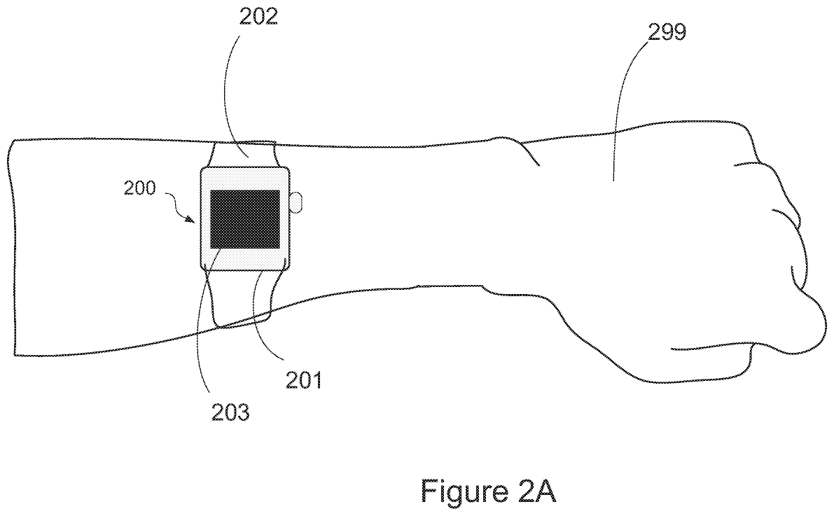

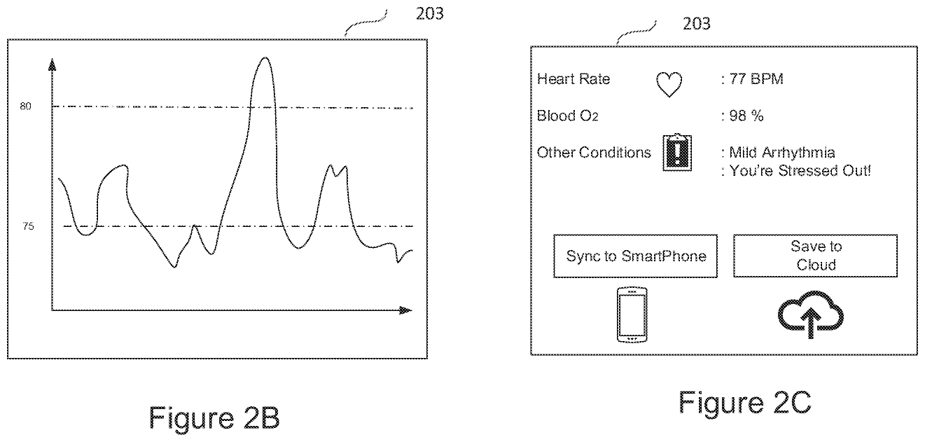

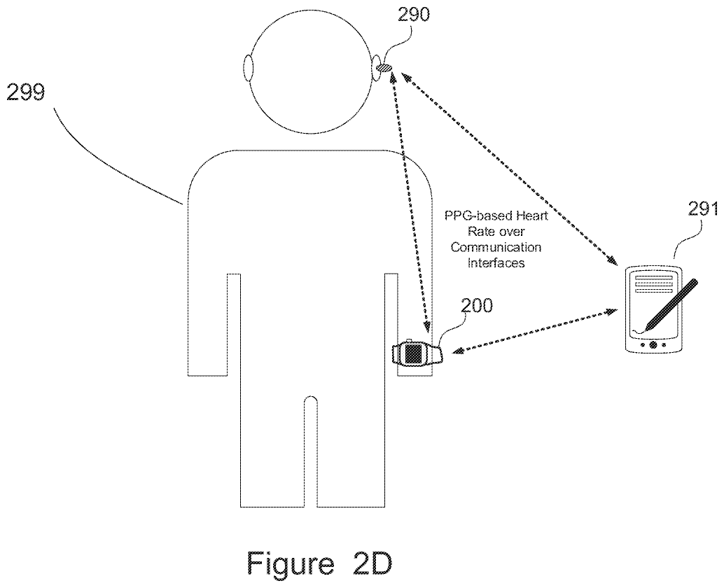

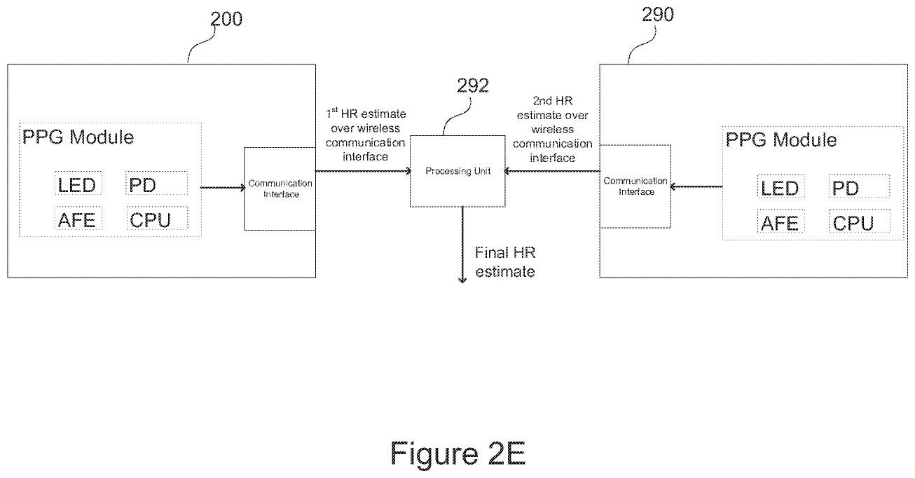

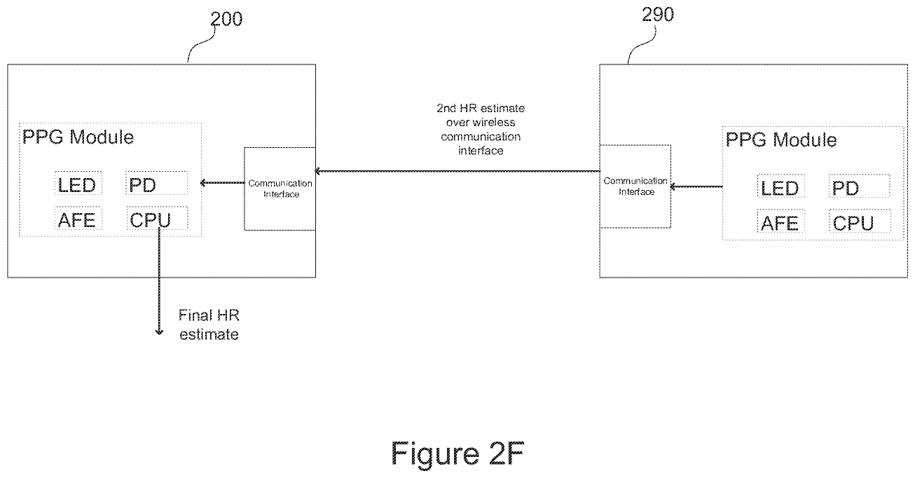

FIG. 2A illustrates a user device, 200, which can be worn by a user, such as user 299. The user device can include a housing 201, and a strap 202. Housing 201 can have components such as a back portion, which will contact the skin of user 299. The back portion can contain a glass portion which will allow light to pass through the back portion. For example, light can be generated from other components contained within housing 201, such as a light source. User device 200 and housing 201 can also have a user interface which allows user 299 to interact and view information from user device 200. The user interface can be part of a touchscreen or other device. Additional components which can be included in user device 200 or in housing 201 are further described above with reference to FIGS. 1A and 1B. The housing can further be of an appropriate thickness to include the components described in FIGS. 1A and 1B. Strap 202 can be a strap to hold the user device on a user, such as one made from metal, leather, cloth, or other material. User device 200 can contain PPG module 100 to perform PPG related functions.

Although a smartwatch is illustrated as user device 200, a person of skill in the art will appreciate that user device 200 can take on a variety of forms. User device 200 can be a smartwatch, a health sensor, an earbud or earplug, headphone, or other wearable electronics, a ring, a bangle, an anklet, necklace, or other piece of jewelry.

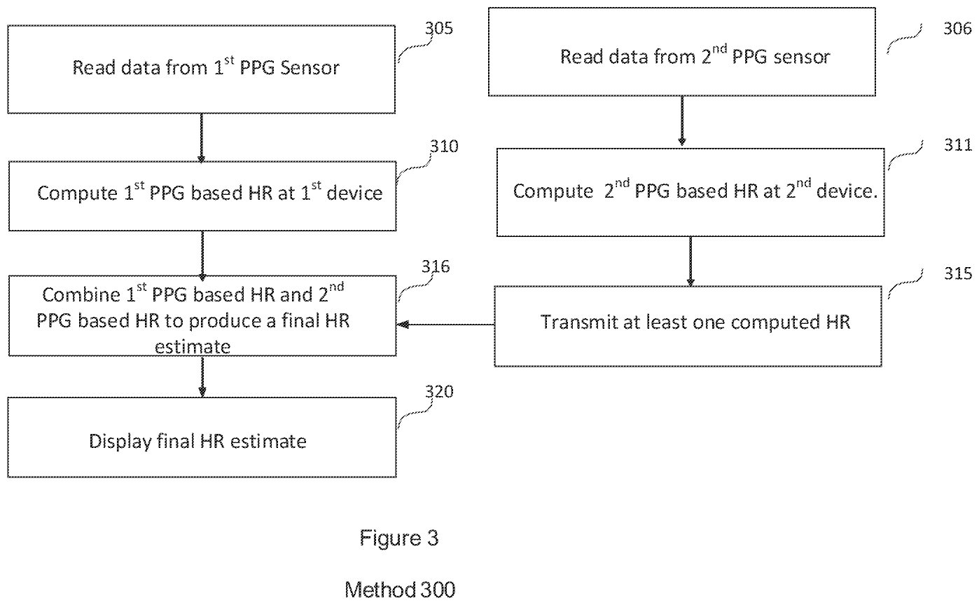

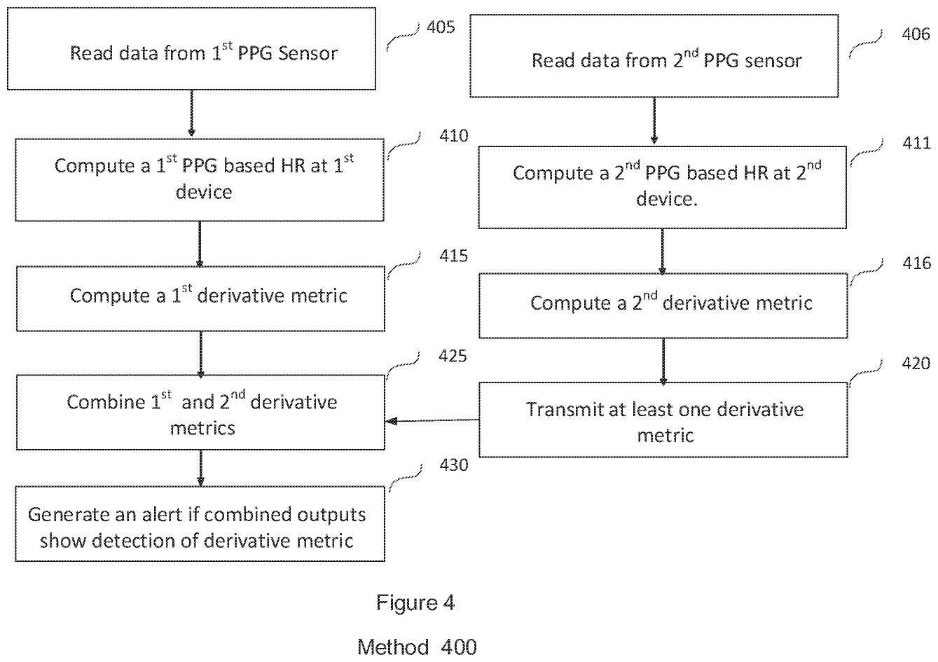

FIG. 2B and FIG. 2C illustrate example formats of displaying information about a physical condition of a user on a display 203. Display 203 can be similar to display 295 described above. FIG. 2B illustrates a graph of the heart rate of a user of a device, such as device 200. This graphical view can be updated in real time to display a trailing number of seconds of the heartbeat of the user. In some examples, in “real time” or “real-time” can mean the execution of data instructions, or algorithms in a short time period, which can provide near-instantaneous output to a user or user device. The heartbeat being displayed can be obtained from the methods described below with reference to FIGS. 3 and 4. FIG. 2C illustrates displaying information about a physical condition of a user in a textual format. For example, FIG. 2B illustrates the current heart rate in beats-per-minute (BPM), the current blood oxygen saturation level, and any other conditions that may be of value to the user, such as an arrhythmia. Although the examples given are for cardiovascular conditions, other aspects of the heart can be monitored. FIG. 2C also illustrates other options, such as the ability to sync the information to another user device, such as a smartphone, or saving the information to another storage unit, such as the internet or to the cloud. Although information is displayed in a visual format, in other examples, the information may be provided through an auditory method. Information being displayed in FIG. 2C can be derived from the methods described below with respect to FIGS. 3 and 4.

FIG. 2D illustrates communication between two user devices, user device 200 and user device 290 worn by user 299. In this instance, user device 290 is an earbud. Although the same reference numerals are used for the devices in FIGS. 2D and 2E as the devices in FIGS. 2A-2C, these devices need not be the same devices. In other examples, user device 290 can be headphones, a pendant, or other device containing a PPG module, such as PPG module 100. As explained further below, each user device can compute a PPG-based heart rate. The computed PPG-based heart rate estimate can be transmitted from one device to another, and the combination of estimates used to improve PPG estimates. In some examples, a non-PPG device, such as smartphone 291 can receive PPG-based heart rate estimates from user device 200 and user device 290 and combine the two estimates together.

FIG. 2E illustrates a schematic view of an example system involving two PPG devices with PPG modules, user device 200 and user device 290, and a processing unit 292. User device 200 and user device 290 can be configured as explained above. Processing unit 292 can be part of smartphone 291. In other examples, processing unit 292 can be part of another user device, such as a smartphone, laptop, desktop computer, or other device capable of receiving wireless communication from the communication interfaces of user device 200 and user device 290. Processing unit 292 can be configured to receive a 1st HR estimate and a 2nd HR estimate from the first user device and the second user device respectively. Processing unit 292 can be similar to the processor(s) 191 described above.

FIG. 2F illustrates a schematic view of an example system involving two PPG devices with PPG modules, user device 200 and user device 290. In this example, user device 290 can transmit a HR estimate calculated at user device 290 via a communication interface to user device 200. User device 200 can use the received HR estimate and combine it with a HR estimate calculated or derived at user device 200. The CPU or other processor of user device 200 can combine the two heart rates to produce a final HR estimate. A person of skill in the art would understand that other configurations of the user devices are possible. For example, user device 200 can send an estimate to user device 290. In other examples, additional user devices can be in communication with one another to provide additional heart rate estimates which can all be used in computation of a final HR estimate.

Further, the transfer of a first heart rate estimate signal and second heart rate estimate signal can include not only a single piece of information being transferred between the device, but rather a continuous or near-continuous signal being transmitted which includes time-series or other information. The first heart rate estimate signal and the second heart rate estimate signal can also be already processed at the first device and the second device respectively. In other examples, other processed signals can be transmitted, such as derivative metrics which are derived from the first rate estimate signal or the second heart rate estimate signal respectively.

### Example Methods

As explained below, the following methods can be used to improve the accuracy of PPG systems by combining a network of PPG nodes into a single heart rate computation that is on average of higher accuracy than any of the individual nodes on its own. At least one of a first PPG system and a second PPG system can be in wireless communication with a CPU that receives a first estimated heart rate from the first PPG system and a second estimated heart rate from the second PPG system. The first and second heart rates are then combined to produce a final heart rate. In some examples, the CPU or processing unit that receives the first estimated heart rate and the second estimated heart rate can be embedded in the first device or the second device, or in a third device that is in communication with the first device, the second device, or both devices. In some examples, a single processor on a first device computes the 1st HR on that device, receives a second HR from a second device, and combines the 1st and 2nd heart rates to produce a final heart rate.

In addition, the following methods can be used in a system to take PPG-based heart rate estimates as inputs into a derivative algorithm, and improve the accuracy of outputs of the derivative algorithm. In some examples, user devices can include PPG nodes and memory with algorithms to compute a derivative metric from a PPG based heart rate using a derivative algorithm. For example, the devices can produce a derivative metric such as the presence of atrial fibrillation (Afib). Atrial fibrillation is an irregular heart rate that can cause a fast and irregular heart rhythm.

In an example, a first PPG system is embedded in a wrist-worn wearable device, and a second PPG sensor is embedded in an ear-worn wearable device. The first and second PPG systems are in communication with a CPU that receives a first estimated heart rate from the first PPG system and a second estimated heart rate from the second PPG system. The first and second heart rate are then combined to produce a final heart rate. The CPU can be contained on either of the devices, or be external to both devices, such as in a smartphone or other device.

FIG. 3 illustrates an example method 300. Method 300 can be used to derive a heart rate which is more accurate and less susceptible to motion artifacts and other errors which can be easily introduced into HR estimates.

At block 305, data can be read from a first PPG sensor. At this block, data can also be read from a second PPG sensor. As explained above with reference to FIG. 1A, a PPG sensor can be embedded into a PPG module of a user device and obtain data by sensing light on the PPG sensor.

At block 310, a 1st PPG based HR can be computed at the first device. The calculation of the 1st PPG heart rate can occur locally at the first device. For example, the PPG heart rate estimate can be calculated at electronics 199 of PPG module 100, which can be embedded into the first device.

At block 311, a second PPG heart rate can also be computed at a second device. The computation of PPG based HR estimates at block 310 and 311 can occur simultaneously, close in time to one another, or in “real-time” with one another.

At block 315, one of the computed heart rates can be transmitted to a user device. In some examples, the 1st PPG based HR computed at the first device can be transmitted to the second device. In other examples, the 2nd PPG based HR computed at the second device can be transmitted to the 1st device. In other examples, the 1st PPG based HR and the 2nd PPG based HR can be transmitted to a third device which does not contain a PPG node.

At block 316, the 1st PPG based HR and the 2nd PPG based HR can be combined to produce a final HR estimate. Block 316 can occur at a PPG node, or a device not containing a PPG node.

The first and second heart rates can be combined by various fusion techniques. One example technique is to use a weighted average whose scales are proportional to the level of confidence of the heart rate output. For example, the confidence level can be based on accelerometer data or accelerometer readings. In some examples, the confidence level can be based on more than one factor, such as the signal to noise ratio or the magnitude of a detected peak. A person of skill in the art will recognize that confidence levels can be estimated or calculated in a variety of ways. Another example technique is to combine the signals using a multivariate kalman filter or extended kalman filter.

An example formula for a weighted combination is shown below:

HR_{final}(n)=ω₁(n)·HR₁(n)+ω₂(n)·HR₂(n)

where:
ω₁(n) is a weight proportional to the confidence of heart rate from the first device
HR₁(n) is the estimated heart rate from the first device
ω₂(n) is a weight proportional to the confidence of heart rate from the second device
HR₂(n) is the estimated heart rate from the second device.

In some examples, n can be a “sample index” where samples are taken at discrete time intervals. In other examples, n can be “time” which is sampled continuously, near-continuously, or at fixed intervals.

In some examples, the weights can be static. In other examples, the weights can be based on historical accuracy information. In other examples, the weights can be changing dynamically.

At block 320, the final HR estimate can be displayed to the user. In some examples, the HR can be provided in other auditory methods, such as through beeps or by using text-to-speech to provide the final HR estimate to the user through synthesized speech. For example, the final HR estimate can be displayed on display 203 of user device 200.

FIG. 4 illustrates an example method 400. In some examples, method 400 can be used to improve the accuracy of derivative metrics. Although method 400 is illustrated with respect to the presence of Afib, a person of skill in the art should recognize that the accuracy of other derivative metrics based on other derivative algorithms can be improved by using method 400. For example, non-limiting examples of derivative metrics sleep analysis, emotion measurement, menstrual cycles tracking, respiration tracking, illnesses detection, hydration analysis.

At block 405, data can be read or obtained from a first PPG sensor. The first PPG sensor can be on a first device.

At block 406, data can be read or obtained from a second PPG sensor. The second PPG sensor can be on a second device.

At block 410, a first PPG based heart rate or heart rate estimate can be generated or computed on the 1st device. The estimate can be based on the data from the first PPG sensor.

At block 411, a second PPG based heart rate or heart rate estimate can be generated or computed on the 2nd device. The estimate can be based on the data from the second PPG sensor.

At block 415, a 1st derivative metric can be computed. The 1st derivative metric can be based on a derivative algorithm which can be stored on the memory of the first device. In some examples, a different derivative metric can be obtained using another derivative algorithm. For example, the derivative metric can be Afib detection. In other examples, the derivative metric can be sleep tracking. In some examples, more than one derivative metric can be calculated. In some examples, more than one derivative metric can be calculated simultaneously or near-simultaneously at the device.

At block 416, a 2nd derivative metric can be computed. The 2nd derivative metric can be the same metric as the 1st derivative metric but be computed on the second device. For example, the derivative metric can be Afib detection.

At block 420, at least one derivative metric can be transmitted to another device. In some examples, the transmission can occur wirelessly.

At block 425, the first derivative metric and the second derivative metrics can be combined. Block 425 can occur at a PPG node, or a device not containing a PPG node. In some examples, the combination can be based on the type of metric being combined. For example, in some examples, the derivative metrics may be critical metrics, such as Afib. In other examples, the derivative metrics, may be metrics related to the user which are not critical, such as sleep tracking or hydration levels. The particular method of combining the metrics can depend on the type of underlying metric.

For example, if a particular derivative metric has a false-positive rate, the metrics can be combined in a manner to reduce the number of false positives associated with the metric. In some examples, two devices, such as a smartwatch and an earbud are detecting a derivative metric independently.

For example, the derived metric can be one with a binary state, such as positive or negative. For example, the derived metric can be related to atrial fibrillation, with a “positive” and “negative” state, and with a false positive rate of 0.1% a day. In some examples, this algorithm could run on a first device such as a smartwatch. Thus, if the algorithm were to be run at a rate of one second, then, over a day, the algorithm would run 86,400 times. At a 0.1% false positive rate, on average, 86.4 false positives would be generated. However, by combining a second atrial fibrillation metric running on a second device, such as earbuds, a much smaller number of false positives can be generated. If the second atrial fibrillation has the same 0.1% false positive rate, it would generate 86.4 false positives. Assuming an independent probability of the false positive rates, when combining the first atrial fibrillation and second atrial fibrillation metric to generate a combined metric only when both atrial fibrillation metrics indicate a “positive” condition at the same time, or within a few second of each other, the total number of false positives would be reduced from 86.4 to 0.086, a reduction of three orders of magnitude. For example:

3600 inspections/hr*24 hrs=86400 inspections per day

0.001 smartwatch false positive rate*0.001 earbud false positive rate=0.000001

86400 inspections*0.000001 false positive rate=0.0864 false positives per day

In contrast, rather than roughly 3 to 4 false positives per hour when using a single PPG measurement, only one false positive would be generated every 11 days when using two PPG nodes. This number can further be decreased if using an additional PPG node. Thus, the combination of both metrics enables an enhanced user experience which is more reliable for detection of underlying medical issues.

In other examples, the derived metrics can be ones that are not binary but rather can be expressed as a range or as a numerical value. For example, the derived metric can be a probability that a certain condition is present or the magnitude of a certain underlying condition. The metrics can be combined using a weighted average. In some examples, an underlying condition related to the metric, such as afib, sleep deprivation, or dehydration, is deemed to be detected if the result of the weighted average of the related derived metrics crosses a predetermined threshold. In some examples, the weights can be static. In other examples, the weights assigned to both can be dynamically adjusted based on factors such as the confidence level of the PPG heart rate estimate that was used as input to the derivative algorithm, or based on detected motion by the accelerometer associated with the PPG module.

In other examples, independent energy expenditure estimates from each device could be combined to reduce overall error and improve overall accuracy. Similarly, other algorithms that perform sleep analysis, measure emotions, track menstrual cycles, track respiration, detect illnesses, analyze hydration, can be improved using the system and methods described above.

In addition, although examples have been given with reference to HR estimates and metrics derived from HR estimates, a person of skill in the art should recognize that the same techniques can be applied to other metrics derivable from the PPG module, such as blood oxygenation saturation (SPO2).

While this disclosure contains many specific implementation details, these should not be construed as limitations on the scope of what may be claimed, but rather as descriptions of features specific to particular implementations. Certain features that are described in this specification in the context of separate implementations may also be implemented in combination in a single implementation. Conversely, various features that are described in the context of a single implementation may also be implemented in multiple implementations separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous.

References to “or” may be construed as inclusive so that any terms described using “or” may indicate any of a single, more than one, and all of the described terms. The labels “first,” “second,” “third,” and so forth are not necessarily meant to indicate an ordering and are generally used merely to distinguish between like or similar items or elements.

Various modifications to the implementations described in this disclosure may be readily apparent to those skilled in the art, and the generic principles defined herein may be applied to other implementations without departing from the spirit or scope of this disclosure. Thus, the claims are not intended to be limited to the implementations shown herein, but are to be accorded the widest scope consistent with this disclosure, the principles and the novel features disclosed herein.

Non-limiting Aspects of the disclosed technology may be represented as the following combination of features:
1. An apparatus, comprising:
   a processing device coupled to a memory storing instructions, the instructions causing the processing device to:
      receive a first heart rate estimate signal from a first wearable device having a first sensor that can generate a first signal associated with a human heart rate,
      receive a second heart rate estimate signal from a second wearable device having a second sensor that can generate a second signal associated with the human heart rate, and
      combine the first heart rate estimate signal and the second heart rate estimate signal to generate a combined heart rate estimate.
2. The apparatus of ¶1, wherein the processing device is configured to receive blood oxygen levels.
3. The apparatus of any one of ¶¶1 through 2, wherein the first sensor and the second sensor are photoplethysmography (PPG) sensors.
4. The apparatus of any one of ¶¶1 through 3, wherein the first wearable device and the second wearable device contain an accelerometer.
5. The apparatus of any one of ¶¶1 through 4, wherein the first heart rate estimate signal and the second heart rate estimate signal are calculated by the first wearable device and the second wearable device based upon data from PPG sensors and accelerometers.
6. The apparatus of any one of ¶¶1 through 3, wherein the processing device is configured to wirelessly receive either the first heart rate estimate signal or the second heart rate estimate signal.
7. The apparatus of any one of ¶¶1 through 6, wherein the processing device is included in the first wearable device.
8. The apparatus of any one of ¶¶1 through 7, wherein the second wearable device wirelessly transmits the second heart rate estimate signal to a first wireless interface included on the first wearable device.
9. The apparatus of any one of ¶¶1 through 8, wherein the first wearable device and the second wearable device comprise either a smartwatch or an earbud.
10. The apparatus of any one of ¶¶1 through 7, wherein the combined heart rate estimate comprises a weighted combination of the first heart rate estimate signal and the second heart rate estimate signal.
11. The apparatus any one of ¶¶1 through 7, wherein the processing device is a smartphone.
12. The apparatus any one of ¶¶1 through 7, comprising a display coupled to the processing device, the processing device configured to notify a user of the combined heart rate estimate signal using the display.
13. The apparatus any one of ¶1, wherein the processing device is configured to generate a third metric based upon a first metric and a second metric, the first metric and the second metric being generated at the first wearable device and the second wearable device respectively, the first metric and the second metric being derived using a derivative algorithm, the derivative algorithm taking as inputs either the first heart rate estimate signal or the second heart rate estimate signal and outputting either the first metric or the second metric.
14. The apparatus any one of ¶13, wherein the third metric is used for one of: atrial fibrillation detection, heart regularity, sleep analysis, emotional measurement, menstrual cycles tracking, respiration tracking, illnesses detection, or hydration levels.
15. The apparatus any one of ¶13 through 14, wherein the third metric is based on a weighted average of the first metric and the second metric.
16. The apparatus of any one of ¶¶13 through 15, wherein the weights are based on a confidence level associated with the first and second heart rate signal.
17. The apparatus of any one of ¶¶13 through 16 wherein the confidence level is based on a motion detection of an accelerometer reading of the first wearable device or the second wearable device or wherein the first metric and the second metric are based on a multivariate Kalman filter.
18. A method of determining a physical condition of a user, the method comprising:
receiving, from a first device, a first measure of a heart rate or a blood oxygenation level of the user;
receiving, from a second device, a second measure of the heart rate or the blood oxygenation level of the user;
generating, at a processing device, a third measure of the heart rate or the blood oxygenation level of the user based on at least a combination of the first measure and the second measure of the heart rate or the blood oxygenation level of the user;
   wherein:
      the first measure of the heart rate or the blood oxygenation level is generated at the first device and is based on at least a first signal received by a sensor of the first device; and
      the second measure of the heart rate or the blood oxygenation level is generated at the second device and is based on at least a second signal received by a sensor of the second device.
19. The method of any one of ¶18, comprising notifying the user of the third measure.
20. The method of any one of ¶18, wherein either the first measure or the second measure is received by the first device or second device, respectively, in real time.
21. The method of any one of ¶¶18 through 20, comprising generating the third measure in real time.
22. The method of any one of ¶¶18 through 21, wherein the processing device is not one of the first device or the second device.
23. The method of any one of ¶¶18 through 21, wherein the processing device is one of either the first device or the second device.
24. The method of any one of ¶¶18 through 23, wherein the processing device applies weights to the first measure and the second measure to generate the third measure.
25. The method of any one of ¶¶18 through 24, wherein the first device and the second device are configured to generate a first derivative metric related to the user and a second derivative metric related to the user respectively using a derivative algorithm.
26. The method of any one of ¶¶18 through 25, wherein the first metric or the second metric are used to determine one of atrial fibrillation detection, heart regularity, sleep analysis, emotional measurement, menstrual cycles tracking, respiration tracking, illnesses detection, or hydration levels.
27. The method of any one of ¶¶18 through 25, wherein the derivative algorithm takes as input the first measure or the second measure and provides as output the first derivative metric or the second derivative metric.
28. The method of any one of ¶¶25 through 27, further comprising the processing device configured to generate a third derivative metric related to the user based upon at least the first derivative metric and the second derivative metric.
29. The method of any one of ¶¶25 through 28, wherein the third metric is not directly calculated from a derivative algorithm.
30. The method of any one of ¶¶26 through 29, comprising providing an alert to the user upon the third metric being generated.
31. The method of any one of ¶26 wherein the third metric is generated upon the first metric and the second metric being generated.
32. A system, comprising:
   a first wearable device having a first wireless interface and a first sensor, the first wearable device configured to generate a first heart rate estimate signal associated with a human heart rate;
   a second wearable device having a second wireless interface and a second sensor, the second wearable device configured to generate a second heart rate estimate signal associated with a human heart rate and; and
   a processing device coupled to a memory storing instructions, the instructions causing the processing device to:
      receive the first heart rate estimate signal from the first wearable device,
      receive the second heart rate estimate signal from a second wearable device, and
      combine the first heart rate estimate signal and the second heart rate estimate signal to generate a combined heart rate estimate.
33. The system of ¶32, wherein the first sensor and the second sensor are photoplethysmography (PPG) sensors.
34. The system of ¶32, wherein the first heart rate estimate signal and the second heart estimate rate signal are calculated by the first wearable device and the second wearable device based upon data from the PPG sensors respectively.
35. The system of ¶34, wherein the processing device is included in the first wearable device and the processing device receives the second heart estimate rate signal using the first wireless interface included on the first wearable device.
36. The system of ¶32, wherein the second wearable device wirelessly transmits the second heart estimate rate signal to the first wireless interface.
37. The system of any one of ¶¶32 through 36, wherein the processing device comprises a smartphone.
38. The system of any one of ¶¶32 through 37, wherein the first wearable device and the second wearable device comprise either a smartwatch or an earbud.

1. An apparatus, comprising:
a processing device coupled to a memory storing instructions, the instructions causing the processing device to:
receive a first heart rate estimate signal from a first wearable device having a first sensor that can generate a first signal associated with a human heart rate,
receive a second heart rate estimate signal from a second wearable device having a second sensor that can generate a second signal associated with the human heart rate, and
combine the first heart rate estimate signal and the second heart rate estimate signal to generate a combined heart rate estimate.

2. (canceled)

3. The apparatus of claim 1, wherein the first sensor and the second sensor are photoplethysmography (PPG) sensors.

4. The apparatus of claim 3, wherein the first wearable device and the second wearable device contain an accelerometer.

5. The apparatus of claim 4, wherein the first heart rate estimate signal and the second heart rate estimate signal are calculated by the first wearable device and the second wearable device based upon data from PPG sensors and accelerometers.

6. (canceled)

7. The apparatus of claim 1, wherein the processing device is included in the first wearable device.

8. The apparatus of claim 7, wherein the second wearable device wirelessly transmits the second heart rate estimate signal to a first wireless interface included on the first wearable device.

9. The apparatus of claim 8, wherein the first wearable device and the second wearable device comprise either a smartwatch or an earbud.

10. (canceled)

11. (canceled)

12. (canceled)

13. The apparatus of claim 1, wherein the processing device is configured to generate a third metric based upon a first metric and a second metric, the first metric and the second metric being generated at the first wearable device and the second wearable device respectively, the first metric and the second metric being derived using a derivative algorithm, the derivative algorithm taking as inputs either the first heart rate estimate signal or the second heart rate estimate signal and outputting either the first metric or the second metric.

14. The apparatus of claim 13, wherein the third metric is used for one of: atrial fibrillation detection, heart regularity, sleep analysis, emotional measurement, menstrual cycles tracking, respiration tracking, illnesses detection, or hydration levels.

15. The apparatus of claim 13, wherein the third metric is based on a weighted average of the first metric and the second metric.

16. The apparatus of claim 15, wherein the weights are based on a confidence level associated with the first and second heart rate signal.

17. The apparatus of claim 16 wherein the confidence level is based on a motion detection of an accelerometer reading of the first wearable device or the second wearable device.

18. (canceled)

19. A method of determining a physical condition of a user, the method comprising:
receiving, from a first device, a first measure of a heart rate or a blood oxygenation level of the user;
receiving, from a second device, a second measure of the heart rate or the blood oxygenation level of the user;
generating, at a processing device, a third measure of the heart rate or the blood oxygenation level of the user based on at least a combination of the first measure and the second measure of the heart rate or the blood oxygenation level of the user;
wherein:
the first measure of the heart rate or the blood oxygenation level is generated at the first device and is based on at least a first signal received by a sensor of the first device; and
the second measure of the heart rate or the blood oxygenation level is generated at the second device and is based on at least a second signal received by a sensor of the second device.

20. (canceled)

21. (canceled)

22. (canceled)

23. (canceled)

24. (canceled)

25. (canceled)

26. The method of claim 19, wherein the first device and the second device are configured to generate a first derivative metric related to the user and a second derivative metric related to the user respectively using a derivative algorithm.

27. The method of claim 26, wherein the first metric or the second metric are used to determine one of atrial fibrillation detection, heart regularity, sleep analysis, emotional measurement, menstrual cycles tracking, respiration tracking, illnesses detection, or hydration levels.

28. The method of claim 26, wherein the derivative algorithm takes as input the first measure or the second measure and providing as output the first derivative metric or the second derivative metric.

29. The method of claim 28, further comprising the processing device configured to generate a third derivative metric related to the user based upon at least the first derivative metric and the second derivative metric.

30. (canceled)

31. The method of claim 29, comprising providing an alert to the user upon the third derivative metric being generated.

32. The method of claim 29 wherein the third metric is generated upon the first metric and the second metric being generated.

33. A system, comprising:
a first wearable device having a first wireless interface and a first sensor, the first wearable device configured to generate a first heart rate estimate signal associated with a human heart rate;
a second wearable device having a second wireless interface and a second sensor, the second wearable device configured to generate a second heart rate estimate signal associated with a human heart rate and; and
a processing device coupled to a memory storing instructions, the instructions causing the processing device to:
receive the first heart rate estimate signal from the first wearable device,
receive the second heart rate estimate signal from a second wearable device, and
combine the first heart rate estimate signal and the second heart rate estimate signal to generate a combined heart rate estimate.

34. (canceled)

35. (canceled)

36. (canceled)

37. (canceled)

38. (canceled)

39. (canceled)
